(19) United States
(12) Patent Application Publication
(10) Pub. No.: US 2009/0171219 A1
(43) Pub. Date: Jul. 2, 2009

(54) BIOPSY GUIDE MOUNTING STRUCTURE, ULTRASONIC PROBE, AND ULTRASONIC DIAGNOSTIC APPARATUS

(76) Inventor: Masami Uchibori, Tokyo (JP)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(21) Appl. No.: 12/339,797

(22) Filed: Dec. 19, 2008

(30) Foreign Application Priority Data
Dec. 27, 2007 (JP) 2007-337122

(51) Int. Cl. A61B 8/00; A61B 10/00

(52) U.S. Cl. 600/461

(57) ABSTRACT

A biopsy guide mounting structure mounts a biopsy guide to guide and hold a biopsy needle to an ultrasonic probe. The biopsy guide mounting structure includes a guide mounting portion provided at a side face of an end portion of the ultrasonic probe. The end portion has an ultrasonic wave transmission/reception surface. The biopsy guide is attached to the guide mounting portion detachably.

### CROSS REFERENCE TO RELATED APPLICATIONS

This application claims the benefit of Japanese Patent Application No. 2007-337122 filed Dec. 27, 2007, which is hereby incorporated by reference in its entirety.

### BACKGROUND OF THE INVENTION

The subject matter disclosed herein relates to a biopsy guide mounting structure for mounting a biopsy guide to guide and hold a biopsy needle to an ultrasonic probe, as well as an ultrasonic probe and an ultrasonic diagnostic apparatus.

Biopsy technique has been adopted wherein a biopsy needle is pierced into a living body and a tumor is cauterized or a tissue is sampled. According to this biopsy technique, biopsy is performed while checking a tomographic image of a pierced region with an ultrasonic diagnostic apparatus. More particularly, an adaptor for biopsy is attached to an ultrasonic probe that performs transmission and reception of an ultrasonic wave, and a biopsy needle is pierced into a living body through a guide hole formed in the adaptor for biopsy while performing ultrasonic imaging.

As the adaptor for biopsy there is disclosed, for example in Japanese Unexamined Patent Publication No. 2007-117128, an adaptor for biopsy including a biopsy guide portion formed with a biopsy needle guide hole to hold a biopsy needle and also including a bracket portion for mounting the biopsy guide portion to an ultrasonic probe.

In the case of the aforesaid adaptor for biopsy provided with both biopsy guide portion and bracket portion, when a biopsy needle pierced into a living body is pulled out from the guide hole, blood adheres to the guide hole and the circumference part thereof. To avoid this inconvenience there has been proposed an adaptor for biopsy wherein the biopsy guide portion and the bracket portion are formed as separate portions so that the biopsy guide portion can be attached to the bracket portion detachably, and the biopsy guide portion is made disposable. However, in the case of such an adaptor for biopsy including the biopsy guide portion and the bracket portion as separate portions, a structure for mounting the biopsy guide portion to the bracket portion is needed, so that when the biopsy guide portion is attached to the ultrasonic probe, the biopsy needle becomes separated from the ultrasonic probe in comparison with the integral type. This results in enlargement of a needle echo-free area, i.e., a biopsy needle blind zone.

### BRIEF DESCRIPTION OF THE INVENTION

It is desirable that the problem described previously is solved.

In a first aspect of the invention there is provided a biopsy guide mounting structure for mounting a biopsy guide to guide and hold a biopsy needle to an ultrasonic probe, the biopsy guide mounting structure including a guide mounting portion provided at a side face of an end portion of the ultrasonic probe, the end portion having an ultrasonic wave transmission/reception surface, the biopsy guide being attached to the guide mounting portion detachably.

In a second aspect of the invention there is provided, in combination with the above first aspect, a biopsy guide mounting structure including the plural guide mounting portions.

In a third aspect of the invention there is provided, in combination with the above second aspect, a biopsy guide mounting structure wherein the mounting angles of the biopsy needles held by the biopsy guides attached to the guide mounting portions respectively are different from one another.

In a fourth aspect of the invention there is provided, in combination with the above third aspect, a biopsy guide mounting structure wherein any of the biopsy guides is attached to the guide mounting portion selected from the plural guide mounting portions so that the mounting angle of the biopsy needle held by the biopsy guide in the ultrasonic probe becomes equal to a desired piercing angle.

In a fifth aspect of the invention there is provided, in combination with the above third aspect, a biopsy guide mounting structure wherein the biopsy guides are attached to the plural guide mounting portions respectively and any of the biopsy needles is held by the biopsy guide which provides a biopsy needle mounting angle in the ultrasonic probe equal to a desired piercing angle.

In a sixth aspect of the invention there is provided, in combination with the above first, second, third, fourth or fifth aspect, a biopsy guide mounting structure wherein each of the guide mounting portions is constituted by a projection formed integrally on a side face of the end portion of the ultrasonic probe, and the biopsy guide has a retaining portion for retaining the projection.

In a seventh aspect of the invention there is provided, in combination with the above sixth aspect, a biopsy guide mounting structure wherein the retaining portion of the biopsy guide is constituted by a pair of holding pieces projecting oppositely to each other from a base portion, the holding pieces being brought into gripping engagement with the projection of the guide mounting portion.

In an eighth aspect of the invention there is provided, in combination with the above seventh aspect, a biopsy guide mounting structure wherein convex portions are formed on opposed surfaces, respectively, of the pair of holding pieces and are retained by concave portions formed in the projection.

In a ninth aspect of the invention there is provided an ultrasonic probe having the mounting structure described in any of the above first to eighth aspects.

In a tenth aspect of the invention there is provided an ultrasonic diagnostic apparatus having the ultrasonic probe described in the above ninth aspect.

According to the invention in the above first aspect, unlike the prior art, since the biopsy guide is mounted directly without a bracket portion to the guide mounting portion provided in the ultrasonic probe, the position of the biopsy needle held by the biopsy guide is closer to the ultrasonic probe in comparison with the case of using a bracket portion. Consequently, the blind zone of the biopsy needle can be made smaller than in the prior art.

According to the invention in the above second aspect, since the plural guide mounting portions are present, the plural biopsy guide can be attached to the ultrasonic probe.

According to the invention in the above third aspect, since the mounting angle of the biopsy needles held by the biopsy guides attached to the guide mounting portions respectively are different, the biopsy needle piercing angle can be made different by suitably selecting any of the biopsy guide mounting portions.

According to the invention in the above fourth aspect, by selecting the guide mounting portion affording a biopsy needle mounting angle in the ultrasonic probe equal to a desired piercing angle and then attaching any of the biopsy guides to the selected guide mounting portion, the biopsy needle piercing angle can be made the desired angle.

According to the invention in the above fifth aspect, since any of the biopsy needles is held by the biopsy guide which provides a needle mounting angle in the ultrasonic probe equal to a desired piercing angle out of the biopsy guides attached to the plural guide mounting portions, the biopsy needle piercing angle can be made the desired angle.

According to the invention in the above sixth aspect, by engagement of the retaining portion of the biopsy guide with the projection, the biopsy guide can be mounted directly to the ultrasonic probe.

According to the invention in the above seventh aspect, by gripping engagement of the holding pieces of the biopsy guide with the projection that constitutes the guide mounting portion, it is possible to mount the biopsy guide.

According to the invention in the above eighth aspect, by engagement of convex portions of the holding pieces with concave portions formed in the projection, it is possible to mount the biopsy guide.

According to the inventions in the above ninth and tenth aspects it is possible to provide an ultrasonic probe and an ultrasonic diagnostic apparatus having the advantages of the inventions in the above first to eighth aspects.

Further objects and advantages of the present invention will be apparent from the following description of embodiments of the invention as illustrated in the accompanying drawings.

### BRIEF DESCRIPTION OF THE DRAWINGS

FIG. 1 is a front view showing an ultrasonic probe having a biopsy guide mounting structure according to an embodiment of the invention.

FIG. 2 is a front view showing a state in which biopsy guides have been removed from the ultrasonic probe shown in FIG. 1.

FIG. 3 is a perspective view of the ultrasonic probe shown in FIG. 2.

FIG. 4 is a perspective view of the ultrasonic probe shown in FIG. 2.

FIG. 5 is an enlarged perspective view of a biopsy guide.

### DETAILED DESCRIPTION OF THE INVENTION

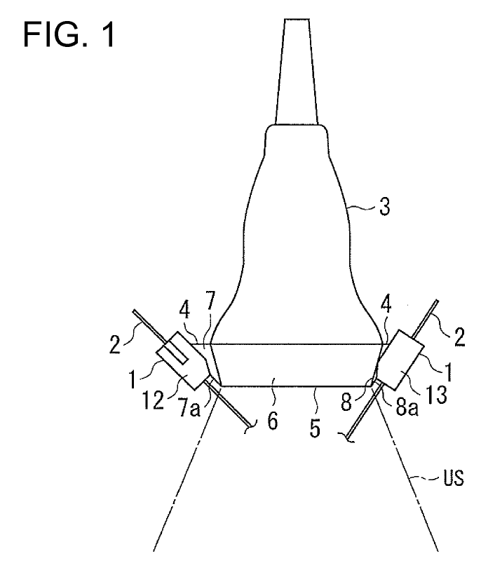

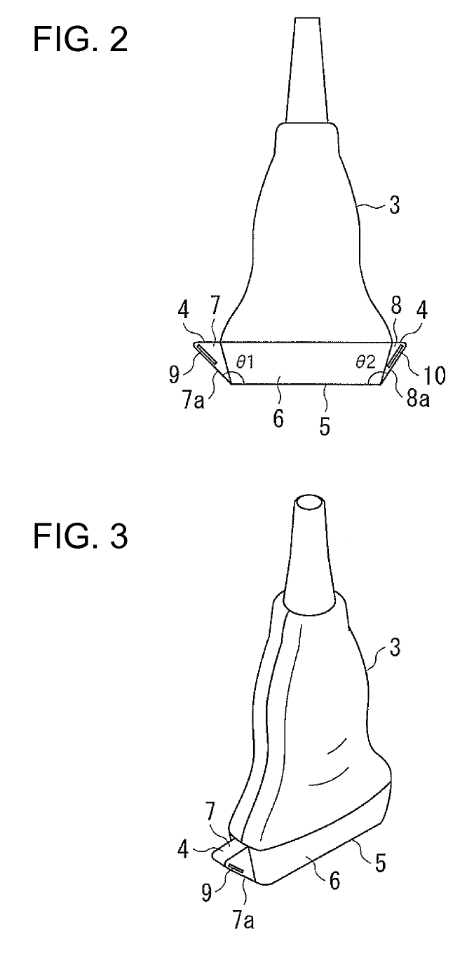

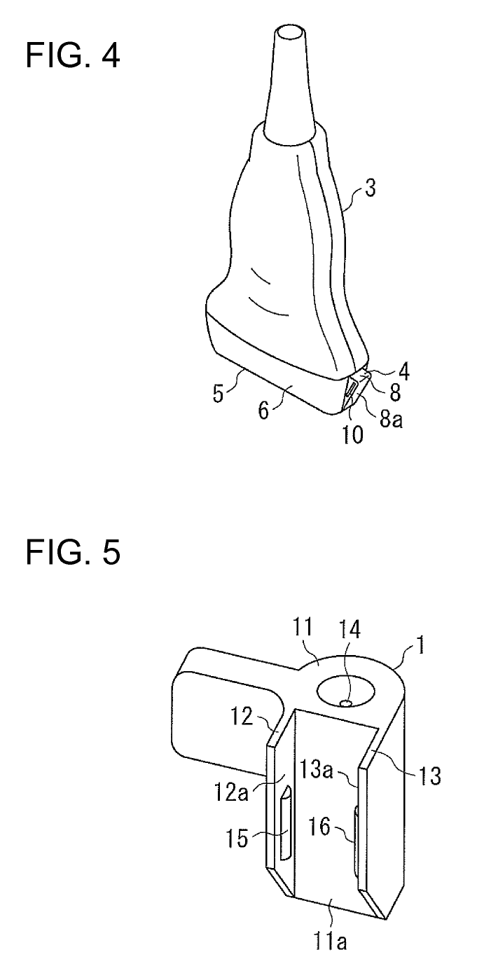

An embodiment of the invention will be described in detail below with reference to the drawings. FIG. 1 is a front view showing an ultrasonic probe having a biopsy guide mounting structure according to an embodiment of the invention, FIG. 2 is a front view showing a state in which biopsy guides have been removed from the ultrasonic probe shown in FIG. 1, and FIGS. 3 and 4 are perspective views of the ultrasonic probe shown in FIG. 2, and FIG. 5 is an enlarged perspective view of a biopsy guide.

In the figures, the numeral 1 denotes a biopsy guide for guiding and holding a biopsy needle 2 and numeral 3 denotes an ultrasonic probe used in an ultrasonic diagnostic apparatus (not shown). The biopsy guide 1 is attached detachably to a guide mounting portion 4 provided in the ultrasonic probe 3.

Two guide mounting portions 4 are provided on side faces of an end portion 6 of the ultrasonic probe 3 which end portion 6 has an ultrasonic wave US transmission/reception surface 5. In this embodiment the guide mounting portions 4 are constituted by a first projection 7 and a second projection 8 which are integral with the side faces of the end portion 6 of the ultrasonic probe 3.

The first and second projections 7, 8 are each in a generally triangular prism shape and formed in the ultrasonic probe 3 so as to be in a generally inverted triangle shape in front view. The angle θ1 (see FIG. 2) between a slant surface 7a of the first projection 7 and the transmission/reception surface 5 and the angle θ2 between a slant surface 8a of the second projection 8 and the transmission/reception surface 5 are different from each other and are in the relation of θ1>θ2.

A pair of concave portions 9 (only one is shown) is formed in opposed surfaces, respectively, of the first projection 7 linearly in parallel with the slant surface 7a. Likewise, a pair of concave portions 10 (only one is shown) is formed in opposed surfaces, respectively, of the second projection 8 linearly in parallel with the slant surface 8a.

The biopsy guide 1 is made up of a base portion 11 and a pair of holding pieces 12 and 13 projecting oppositely to each other from the based portion 11, the holding pieces 12 and 13 being held by the first projection 7 or the second projection 8. The base portion 11 is formed with a guide hole 14 for insertion therein of the biopsy needle 2 and for guiding and holding the same needle.

The pair of holding pieces 12 and 13 is an example of the retaining portion for retaining engagement with the projection that constitutes each guide mounting portion in the invention. The spacing between the holding pieces 12 and 13 is set at such spacing as permits the two to be held by the first projection 7 or the second projection 8.

Convex portions 15 and 16 are formed on opposed surfaces 12a and 13a, respectively, of the holding pieces 12 and 13. The holding pieces 12 and 13 are deformed elastically, causing the convex portions 15 and 16 to be retained by the concave portions 9 of the first projection 7 or the concave portions 10 of the second projection 8. With the holding pieces 12 and 13 thus held by the first projection 7 or the second projection 8, the biopsy guide 1 is mounted to the first projection 7 or the second projection 8.

The biopsy guide 1 is mounted to the first projection 7 or the second projection 8 in a state in which a surface 11a of the base portion 11 for projection of the holding pieces 12 and 13 is abutted against the slant face 7a or 8a of the first projection 7 or the second projection 8. Since the angles θ1 and θ2 between the slant surfaces 7a, 8a and the transmission/reception surface 5 are different from each other, the mounting angle of the biopsy needle 2 held by the biopsy guide 1 can be made different between the case where the biopsy guide 1 is mounted to the first projection 7 and the case where it is mounted to the second projection 8. In this embodiment, as noted above, since the angles θ1 and θ2 are in the relation of θ1>θ2, the biopsy needle 2 held by the biopsy guide 1 attached to the first projection 7 is pierced into a living body at an acuter angle than the biopsy needle 2 held by the biopsy guide 1 attached to the second projection 8.

In order that the mounting angle of the biopsy needle 2 held by the biopsy guide 1 in the ultrasonic probe 3 may become a desired piercing angle, either the first projection 7 or the second projection 8 is selected and the biopsy guide 1 is attached to the selected projection. Alternatively, the biopsy guide 1 is attached to each of the first and second projections 7, 8, then the one affording a mounting angle of the biopsy needle 2 in the ultrasonic probe 3 equal to a desired piercing angle is selected and the biopsy needle 2 is held by the selected biopsy guide.

Further, the biopsy guide 1 may be attached to each of the first projection 7 and the second projection 8, and the biopsy needles 2 may be held by both biopsy guides 1 respectively.

After use of the biopsy needles 2, the biopsy guides 1 attached to the first and second projections 7, 8 can be removed by deforming the holding pieces 12 and 13 elastically.

According to this embodiment, since each biopsy guide 1 is directly mounted respectively to the guide mounting portions 4 in the ultrasonic probe 3 without using such a bracket portion as in the prior art, the position of the biopsy needle 2 held by each biopsy guide 1 becomes closer to the ultrasonic probe 3 in comparison with the case of using a bracket portion. Consequently, the blind zone of the biopsy needle 2 can be made smaller than in the prior art.

Besides, since two guide mounting portions 4 described above are present, two biopsy guides 1 described above can be mounted and it is possible to effect mounting of plural biopsy needles 2 for the ultrasonic probe 3. Further, the mounting angle of the biopsy needle 2 held by each biopsy guide 1 can be made different between the case where the biopsy guide is attached to the first projection 7 and the case where it is attached to the second projection 8.

Although the invention has been described above by way of the above embodiment, it goes without saying that various changes may be made within the scope not departing from the gist of the invention. For example, though not shown, the angles θ1 and θ2 between the slant surfaces 7a, 8a of the first and second projections 7, 8 and the transmission/reception surface 5 may be equal to each other and the mounting angle of the biopsy needle 2 in the ultrasonic probe 3 may be equal between the case where the biopsy needle 2 is held by the biopsy guide 1 attached to the first projection 7 and the case where it is held by the biopsy guide 1 attached to the second projection 8.

Many widely different embodiments of the invention may be configured without departing from the spirit and the scope of the present invention. It should be understood that the present invention is not limited to the specific embodiments described in the specification, except as defined in the appended claims.

1. A biopsy guide mounting structure for use in mounting a biopsy guide configured to guide and hold a biopsy needle to an ultrasonic probe, said biopsy guide mounting structure comprising a guide mounting portion positioned at a side face of an end portion of the ultrasonic probe, the end portion having an ultrasonic wave transmission/reception surface, the biopsy guide being detachably attached to said guide mounting portion.

2. A biopsy guide mounting structure according to claim 1, wherein said guide mounting portion comprises a plurality of guide mounting portions.

3. A biopsy guide mounting structure according to claim 2, wherein a mounting angle of a biopsy needle held by a biopsy guide attached to each of said plurality of guide mounting portions respectively are different from one another.

4. A biopsy guide mounting structure according to claim 3, wherein a biopsy guide is coupled to a corresponding guide mounting portion of said plurality of guide mounting portions such that a mounting angle of the biopsy needle held by the biopsy guide in the ultrasonic probe becomes equal to a desired piercing angle.

5. A biopsy guide mounting structure according to claim 3, wherein the biopsy guides are each coupled to a corresponding guide mounting portion of said plurality of guide mounting portions respectively and a biopsy needle is held by a corresponding biopsy guide in order to provide a biopsy needle mounting angle in the ultrasonic probe equal to a desired piercing angle.

6. A biopsy guide mounting structure according to claim 1, wherein said guide mounting portion comprises a projection formed integrally on a side face of the end portion of the ultrasonic probe, and the biopsy guide includes a retaining portion for retaining the projection.

7. A biopsy guide mounting structure according to claim 2, wherein each of said plurality of guide mounting portions comprises a projection formed integrally on a side face of the end portion of the ultrasonic probe, and each biopsy guide has includes a retaining portion for retaining the projection.

8. A biopsy guide mounting structure according to claim 3, wherein each of said plurality of guide mounting portions comprises a projection formed integrally on a side face of the end portion of the ultrasonic probe, and each biopsy guide includes a retaining portion for retaining the projection.

9. A biopsy guide mounting structure according to claim 4, wherein each of said plurality of guide mounting portions comprises a projection formed integrally on a side face of the end portion of the ultrasonic probe, and each biopsy guide includes a retaining portion for retaining the projection.

10. A biopsy guide mounting structure according to claim 5, wherein each of said plurality of guide mounting portions comprises a projection formed integrally on a side face of the end portion of the ultrasonic probe, and each biopsy guide includes a retaining portion for retaining the projection.

11. A biopsy guide mounting structure according to claim 6, wherein the retaining portion of the biopsy guide includes a pair of holding pieces projecting oppositely to each other from a base portion, the holding pieces being brought into gripping engagement with said projection of said guide mounting portion.

12. A biopsy guide mounting structure according to claim 11, wherein a plurality of convex portions are formed on opposed surfaces, respectively, of the pair of holding pieces such that the plurality of convex portions are retained by concave portions formed in said projection.

13. An ultrasonic probe comprising:
a biopsy needle:
a biopsy guide configured to guide and hold said biopsy needle, and
a biopsy guide mounting structure coupled to said biopsy guide, said biopsy guide mounting structure comprising a guile mounting portion positioned at a side face of an end portion of said ultrasonic probe, said end portion comprising an ultrasonic wave transmission/reception surface, said biopsy guide is detachably attached to said guide mounting portion.

14. An ultrasonic probe according to claim 13, wherein said guide mounting portion comprises a plurality of guide mounting portions.

15. An ultrasonic probe according to claim 14, wherein said biopsy needle comprises a plurality of biopsy needles, said biopsy guide comprises a plurality of biopsy guides, a mounting angle of each of said plurality of biopsy needles held by a corresponding biopsy guide of said biopsy guides coupled to each of said plurality of guide mounting portions respectively are different from one another.

16. An ultrasonic probe according to claim 15, wherein a biopsy guide of said plurality of biopsy guides is coupled to a corresponding guide mounting portion of said guide mounting portions such that the mounting angle of a corresponding biopsy needle of said plurality of biopsy needles held by said biopsy guide in said ultrasonic probe becomes equal to a desired piercing angle.

17. An ultrasonic probe according to claim 15, wherein each of said plurality of biopsy guides is coupled to a corresponding guide mounting portion of said plurality of guide mounting portions such that a corresponding biopsy needle of said plurality of biopsy needles is held by said biopsy guide in order to provide a biopsy needle mounting angle within said ultrasonic probe equal to a desired piercing angle.

18. An ultrasonic probe according to claim 13, wherein said guide mounting portion comprises a projection formed integrally on a side face of said end portion of said ultrasonic probe, said biopsy guide comprises a retaining portion configured to retain said projection.

19. An ultrasonic probe according to claim 14, wherein each of said plurality of guide mounting structures comprises a projection formed integrally on a side face of said end portion of said ultrasonic probe, said biopsy guide comprises a retaining portion configured to retain said projection.

20. An ultrasonic diagnostic apparatus comprising:
an ultrasonic probe comprising:
a biopsy needle:
a biopsy guide configured to guide and hold said biopsy needle; and
a biopsy guide mounting structure coupled to said biopsy guide, said biopsy guide mounting structure comprising a guide mounting portion positioned at a side face of an end portion of said ultrasonic probe, said end portion comprising an ultrasonic wave transmission/reception surface, said biopsy guide is detachably attached to said guide mounting portion.
